(19) United States
(12) Patent Application Publication
(10) Pub. No.: US 2009/0227849 A1
(43) Pub. Date: Sep. 10, 2009

(54) Medical Instrument

(75) Inventors: Daniel A. Goor, Tel Aviv (IL); Evgeny Granov, Ra'anana (IL); Igor Granov, Raanana (IL)

Correspondence Address: HOUSTON ELISEEVA, 4 MILITIA DRIVE, SUITE 4, LEXINGTON, MA, 02421, US

(73) Assignee: N.I. MEDICAL LTD., Kfar Malal (IL)

(21) Appl. No.: 12/397,423

(22) Filed: Mar. 4, 2009

Related U.S. Application Data
(63) Continuation of application No. PCT/IL2007/001087, filed on Sep. 4, 2007.
(60) Provisional application No. 60/842,159, filed on Sep. 5, 2006.

(51) Int. Cl. A61B 5/00

(52) U.S. Cl. 600/301

(57) ABSTRACT

A medical instrument is presented. The medical instrument comprises a housing, a first communication utility in the housing for receiving input signal indicative of physiological signals, and a second communication utility for communicating with a computer. The housing is configured for removable installment in a compact disc drive bay of a computer.

### RELATED APPLICATIONS

This application is a Continuation of PCT application serial number PCT/IL2007/001087, filed on Sep. 4, 2007, which in turn claims the benefit under 35 USC 119(e) of U.S. Provisional Application No. 60/842,159, filed on Sep. 5, 2006, both of which are incorporated herein by reference in their entirety.

### FIELD OF THE INVENTION

The invention concerns a medical instrument that is adapted for receiving physiological signals from the body of a subject and which is adapted for communication with a computer for the purpose of analysis, storage and display of the recorded signal or a processed product thereof.

### BACKGROUND OF THE INVENTION

It is a trend is modern medicine to transfer many of the medical and diagnostic procedures hitherto performed in hospitals or specialized clinics, to a doctor's office. Additionally, there is also a trend to switch medical instruments from a stand-alone configuration into a configuration in which they are integrated with a computer. For this the instrument has to be provided with some means for interfacing with the computer. For a doctor's office use as well as for use in the medical facility without specialized computer technicians, such interfacing should be designed to be relatively simple.

Hitherto, computer-interfacing medical instruments were typically provided as separate units connectable to a computer via one of the computer's standard communication ports.

### SUMMARY OF THE INVENTION

The present invention provides a new design for a medical instrument of the kind that is adapted to receive signals representative of physiological signals and transmit them, typically after initial processing, to a computer. In accordance with the invention such a medical instrument is provided with a housing configured such that it can be installed in a compact disc (CD) drive bay of a computer, typically a portable computer, e.g. of the kind usually referred to as “laptop” or “notebook” computers.

Thus, in accordance with the present invention there is provided a medical instrument comprising a housing, a first communication utility in the housing for receiving input signal indicative of physiological signals and a second communication utility for communicating with a computer, said housing being configured for removable installment in a CR drive bay of a computer.

The term “medical instrument” means to denote any instrument that is intended to record physiological signals and includes, but not limited to, medical instruments intended to record cardiovascular parameters. In addition, the medical instrument may be adapted for recording of brainwaves, parameters of the skin, parameters indicative of tissue composition, sound signals from various body parts, etc. As will be appreciated, the invention is widely applicable and is not limited for the aforementioned medical applications.

The first communication utility comprises, according to an embodiment of the invention, one or more signal receivers, and optionally initial signal conversion or amplifier stage. Also, according to some embodiments of the invention, the medical instrument may also comprise an emitting utility for outputting a signal, e.g. a stimulating signal, to a subject body, such as a signal generator for emitting a low voltage electrical stimulation signal for inducing evoked potentials in nerves or muscles, an ultrasound transducer for emitting an ultrasound signal, a laser for emitting a light signal, and others.

Said second communication utility may, according to one embodiment of the invention, have a port disposed on a face of the housing which is externally accessible when the instrument is in said bay. Said second communication utility, according to such embodiments, may communicate with the computer via a wired or wireless communication connection arrangement.

According to other embodiments of the invention, said second communication utility is on a face which, once the instrument is in said bay, is internal, and is adapted for connection to a communication port that is within said bay, e.g. a communication port that is adapted for communication with standard computer accessories.

According to an embodiment of the invention, said housing is configured for installment in the bay of a portable computer. Typically, such housing is configured for installment in a bay intended for a CD disc-drive. According to an embodiment of the invention, the housing also houses a processor utility for processing the input signal and generating an output signal corresponding thereto for communicating to the computer. According to some embodiments of the invention, said processing utility is adapted also for receiving control signals from the computer. According to other embodiments of the invention, the medical instrument also comprises an application software utility, e.g. a software utility which is intended to permit the computer to integrate with said instrument.

A medical instrument, according to an embodiment of the invention, is adapted for use in determining one or more cardiovascular parameters. The cardiovascular parameters may be one or more of the following: ECG, blood pressure, stroke volume, heart rate or cardiac output.

According to an embodiment of the invention, the first communication utility comprises two or more signal receivers, each for receiving an input signal indicative of a different physiological parameter. According to a specific embodiment, said first communication utility comprises a first receiver for receiving a signal indicative of a parameter relating to cardiac output, a second receiver for receiving a signal indicative of an ECG and a third receiver for receiving a signal indicative of blood pressure.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic illustration of a medical instrument in accordance with an embodiment of the present invention;

FIG. 2A is a block diagram illustrating the main elements within the medical instrument in accordance with an embodiment of the invention and its connectivity to external measurement unit and to a host computer; and

FIG. 2B exemplifies more specifically an embodiment of the bioimpedance unit of the medical instrument of FIG. 2A.

### DETAILED DESCRIPTION OF THE PREFERRED EMBODIMENTS

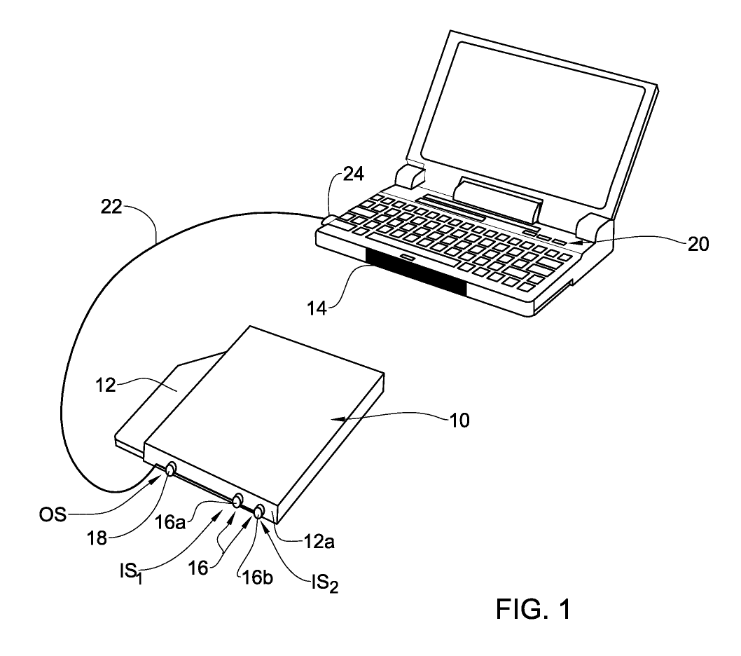

Reference is made to FIG. 1 illustrating schematically an example of a medical instrument 10 according to the present invention. The medical instrument of the present invention is designated as a so-called Medical Gear In Contrivance (MEGIC) device. Instrument 10 includes a housing 12 configured for removable installment in a standard CD drive bay 14 of a computer, e.g. that of a portable computer such as a Laptop. Housing 12 includes a first communication utility 16 for receiving one or more input signals, two such signals IS₁ and IS₂ being considered in the present specific but not limiting example, and includes a second communication utility 18 for communicating with a computer 20. Input signal(s) is/are indicative of physiological signal(s).

It should be noted that generally the first communication utility may be formed by a single port for receiving one or more input signals. In the present example, two such ports 16A and 16B are shown. The use of multiple receiving ports might be associated with concurrent receipt of multiple different input signals, or with a need for selective operation of either one of them, for example when the other is being corrupted, as the case may be.

Second communication utility 18 also may be formed by one or more ports—one such port being shown in the present example. Second communication utility 18 is adapted for communication via a connection unit 22 to a communication port 24 of the computer. In the present example, such a connection unit is a wire-based connector, but it should be understood that wireless communication can be used as well.

In the present example, the ports of first and second communication utilities 16 and 18 are located on a face 12A of the housing which is externally accessible once the instrument is in the computer bay. It should however be noted that at least some of the ports of the first and second communication utilities, typically that associated with connection to the computer's utilities, may be located on a face of the housing which is internal once the instrument is in the bay. A connection base in a computer, such as that known as a laptop or notebook computer, is generally equipped with standard internal communication ports adapted for connection with counterpart connectors in computer accessories which are intended for installment in such ports. According to embodiments of the present invention, the medical instrument may be fitted with corresponding connection ports which then connect into the standardized connection ports within the base.

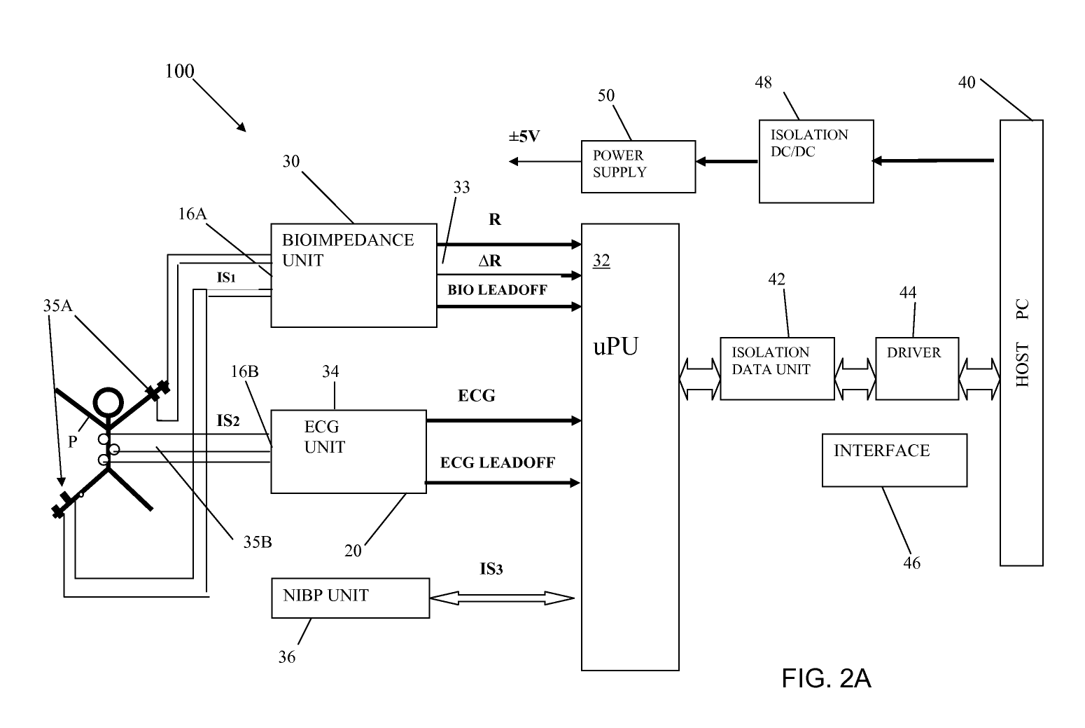

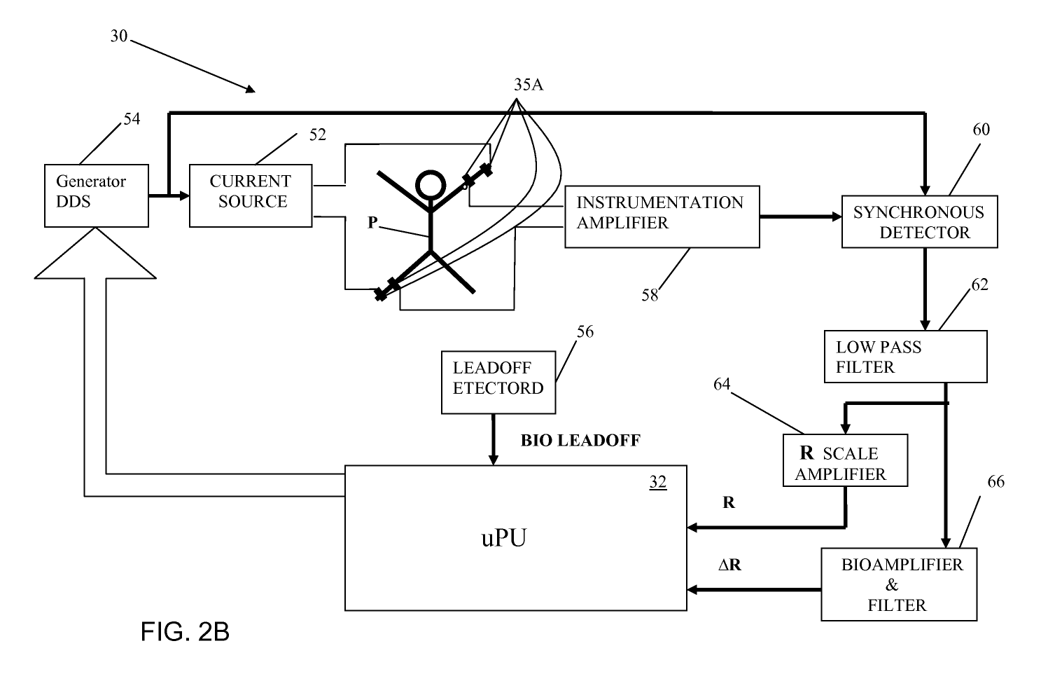

Housing 12 includes a processor utility (not shown in FIG. 1, and described further below with reference to FIG. 2A) for processing the input signal(s) IS₁, IS₂ and generating an output signal OS to the computer. The processor utility features are appropriately set for the requirements of various applications of the invention, for monitoring various physiological parameters of individuals.

In some embodiments of the invention, the processor utility of the medical instrument operates as an application program interface utility between the computer and external measurement unit(s), while the data processing is mainly carried out by the computer. For example, the processor utility can perform the signal amplification, filtering, etc. In some other embodiments, the processor utility is installed with a certain software product operating to perform the entire medical data processing, and the computer serves for displaying the processing results and provides a user interface for operating the medical instrument, data storage, etc. In yet another embodiment of the invention, medical instrument 10 includes an application software utility configured for the so-called distributed data processing in integration with said computer.

In a specific non-limiting example of the invention, medical instrument 10 is adapted for use in determining one or more cardiovascular parameters of a patient. The cardiovascular parameters may be one or more of the following parameters: ECG, blood pressure, stroke volume, heart rate or cardiac output.

Reference is made to FIGS. 2A and 2B showing schematically the internal components of a medical instrument according to an embodiment of the invention. FIG. 2A shows the components of the processor utility 100 of a medical instrument, according to an embodiment of the invention and FIG. 2B shows more specifically a bioimpedance unit of the processor utility.

Processor utility 100 includes a bioimpedance unit 30 constituting a signal receiver, and a microcontroller 32. Bioimpedance unit 30 is connected at its input end to input port 16A of the medical instrument and is connected at its output end 33 to microcontroller 32. Bioimpedance unit 30 is configured for receiving and performing the processing of an electrical output IS₁ from an electrodes' unit 35A on a patient P for determining the patient's cardiac parameters, such as stroke volume. In a non-limiting example, processor utility 100 also includes another signal receiver 34 for receiving, via an input port 16B, an input signal IS₂ from an ECG-related electrodes' unit 35B. In the present example different input signals are received by different input ports, but it should be understood that this is an optional solution, and all the signals can be input through the same port. Also, in this example, the medical instrument receives, through either one of ports 16A and 16B, an input signal IS₃ from a non-invasive blood pressure (NIBP) measurement unit 36 (wire or wireless).

The outputs of microcontroller 32 are connected to an output port (18 in FIG. 1) of the medical instrument connected to a HOST processor 40 of the computer (20 in FIG. 1). This connection may be implemented via an isolation data unit 42 (such as opto-isolators HCPL2611HP), and via a Driver circuit 44 (such as the driver RS232C or USB) and suitable interface utility 46. Alternatively the connection may be through dedicated port in the instrument that connects to a standardized connection port in the bay. These connection elements may be part of the medical instrument and/or the computer. As indicated above, the connection between the medical instrument and the computer may be wire-based or wireless. In the latter case, the medical instrument and the computer are appropriately provided with IR and/or RF and/or acoustic transmitter/receiver. Such communication techniques are known per se and therefore need not be described in detail herein. The medical instrument of the illustrated embodiment is power supplied (e.g. +5V) from the HOST processor via an isolating DC/DC circuit 48 and feeding a power supply unit 50 which stabilizes the voltage value. In the present not limiting example, the processing is a distributed processing, namely bioimpedance unit 30 and local microcontroller 32 perform certain initial processing (as will be described below with reference to FIG. 2B), while HOST processor 40 performs the final processing, which includes analysis of the measured signal, selection of a model (which is stored in a memory utility of either computer 20 or medical instrument 10) for calculation, and calculation of the desired parameter(s). For example, the cardiac parameter(s) may be calculated based on the models utilizing the ΔR/R volumetric parameter and the ((dR/dt)·T) blood velocity parameter. To this end, the known Frinerman formula (see U.S. Pat. No. 5,469,859 assigned to the assignee of the present application) and the dR/dt-based equation [Kubicek et al. Biomed. Eng. 1974, 9:410-16] can be used for the stroke volume calculation. The principles of these bioimpedance measurement techniques are known per se and therefore need not be specifically described.

An example of the construction and operation of bioimpedance unit 30 will now be described with reference to FIG. 2B. As indicated above, this is a specific but not limiting example of the invention configured for use in determination of one or more cardiac parameters of a patient. Bioimpedance unit 30 includes a current source 52 for supplying electrical current to electrodes' unit 35A, and a direct digital synthesizer (DDS) generator 54, which is in turn operated by microcontroller 32. The provision of DDS generator 54 is associated with the following: a human body behaves, from an electrical point of view, as a resistance-capacitance (RC) impedance; the value of impedance is influenced by the injection current frequency; the DDS generator operates to control this frequency to be about 32 KHz. Microcontroller 32 operates the DDS generator to control the stability of the output frequency and amplitude of a sinusoidal signal from the DDS generator, which signal operates current source 52. Bioimpedance unit 30 preferably includes a leadoff detector 56, which is used to sense the absence of a contact between the electrode and the body (for bioimpedance electrodes and preferably also for ECG electrodes).

A read voltage signal IS₁ from electrodes' unit 35A is input to a high precision instrumentation amplifier 58. This input signal IS₁ is proportional to the human body impedance Z (i.e. an integral bioimpedance). An output of instrumentation amplifier 58 is connected to a first input of a synchronous detector 60. The latter operates to rectify the integral bioimpedance signal, and to provide simultaneous derivation of the active component R of the integral bioimpedance signal vector. This component R is directly proportional to the resistive component of the lead (resistance of the blood system). Linearity of the synchronous detector 60 simplifies the calibration process and reduces it to a single-step initial adjustment of the medical instrument (instead of a per cycle calibration). An output of detector 60 is connected to a low frequency filter 62 which may for example be a low pass Bessel filter. Filter 62 cuts off high frequency components, for example above 32 KHz, and delivers an operating signal, which has the active bioimpedance component (DC) R, and the waveform bioimpedance signal (AC) ΔR. The operating signal is input to an R SCALE AMPLIFIER 64 and to a Bioamplifier and Filter 66. Amplifier 64 produces an output signal proportional to the active bioimpedance component R and transmits the same to an input ADC (Analog-to-Digital Converter) of the microcontroller 32. Bioamplifier and Filter 66 separates from the operating signal the waveform ΔR component. The output of the Bioamplifier and Filter 66 is connected to another input ADC of microcontroller 32. The latter communicates with HOST processor (40 in FIG. 2A) of the computer.

Thus, the present invention provides a novel medical instrument configured to be installable in a standard computer bay, typically a disc drive bay. The medical instrument is preferably constructed as an application program interface for communicating with a measurement unit, such as electrode unit(s), and for communicating with a computer processor.

It should be understood that the invention is not limited to the above-described examples of determining cardiovascular parameters, but may be used for determining any other physiological parameters. To this end, the processor utility of the medical instrument is installed with suitable hardware and/or software products to be responsive to predetermined input signals and process them to produce desired output.

While this invention has been particularly shown and described with references to preferred embodiments thereof, it will be understood by those skilled in the art that various changes in form and details may be made therein without departing from the scope of the invention encompassed by the appended claims.

1. A medical instrument comprising a housing, a first communication utility in the housing for receiving input signal indicative of physiological signals and a second communication utility for communicating with a computer, said housing being configured for removable installment in a compact disc (CD) drive bay of a computer.

2. The medical instrument according to claim 1, wherein said first communication utility and said second communication utility has a port on a face of the housing which is externally accessible once the instrument is in said bay, said second communication utility is adapted for communication via a connection unit to a communication port of the computer.

3. The medical instrument according to claim 1, wherein said first communication utility is on a face of the housing which is externally accessible once the instrument is in said bay and said second communication utility is on a face which is internal once the instrument is in said bay and adapted for connection to a communication port that is within said bay.

4. The medical instrument according to claim 1, wherein said housing houses a processor utility for processing the input signal and generating an output signal corresponding thereto for communicating to the computer.

5. The medical instrument according to claim 4, wherein said processor utility is adapted for receiving control signal from the computer.

6. The medical instrument according to claim 4, comprising an application software utility.

7. The medical instrument according to claim 1, for use in determining one or more cardiovascular parameters.

8. The medical instrument according to claim 7, wherein said cardiovascular parameters are selected from ECG, blood pressure, stroke volume, heart rate, cardiac output and cardiac output derivatives.

9. The medical instrument according claim 1, wherein said first communication utility comprise two or more signal receivers each for receiving an input signal indicative of a different physiological parameter.

10. The medical instrument according to claim 9, comprising a first receiver for receiving signal indicative of a parameter relating to cardiac output, a second receiver for receiving a signal indicative of ECG and a third receiver for receiving a signal indicative of blood pressure.
